(19) United States
(12) Patent Application Publication
(10) Pub. No.: US 2014/0301965 A1
(43) Pub. Date: Oct. 9, 2014

(54) CLEANING AGENT HAVING HIGH CONDITIONING EFFECTIVENESS

(71) Applicant: Henkel AG & Co. KGaA, Duesseldorf (DE)

(72) Inventors: Thomas Schroeder, Hamburg (DE); Dirk Hentrich, Hamburg (DE)

(21) Appl. No.: 14/309,964

(22) Filed: Jun. 20, 2014

Related U.S. Application Data
(63) Continuation of application No. PCT/EP2012/074461, filed on Dec. 5, 2012.

(30) Foreign Application Priority Data
Dec. 20, 2011 (DE) 10 2011 089 226.5

(51) Int. Cl. A61K 8/894; A61Q 5/00; A61K 8/46; A61Q 5/02; A61K 8/73; A61K 8/44

(52) U.S. Cl. CPC A61K 8/894; A61K 8/731; A61K 8/442; A61K 8/463; A61Q 5/02; A61Q 5/002; A61Q 5/00
USPC 424/70.13

(57) ABSTRACT

The invention relates to a cosmetic cleaning agent including in a cosmetically acceptable carrier a) at least one anionic surfactant, b) at least one amphoteric and/or zwitterionic surfactant of the following formula (I), in which—R denotes a straight-chain or branched, saturated or mono- or polyunsaturated alkyl or alkenyl residue having 8 to 24 carbon atoms, and—R1 and R2 independently of one another denote the groups —(CH₂)ₙ—COOX, —(CH₂)ₙ—O—(CH₂)ₙ—COOX or —(CH₂)ₙ—OH, wherein n is an integer from 1 to 4 and X is an alkali, an alkaline-earth, an ammonium or an alkanolamine ion, c) at least one silicone of the following formula (II), in which—the residue R denotes a hydrogen atom, an alkyl group having 1 to 12 C atoms, an alkoxy group having 1 to 12 C atoms or a hydroxyl group,—x denotes a number from 1 to 100,—y denotes a number from 1 to 20, and—a and b independently of one another denote numbers from 0 to 50, and d) at least one cationic polymer selected from quaternized cellulose derivatives.

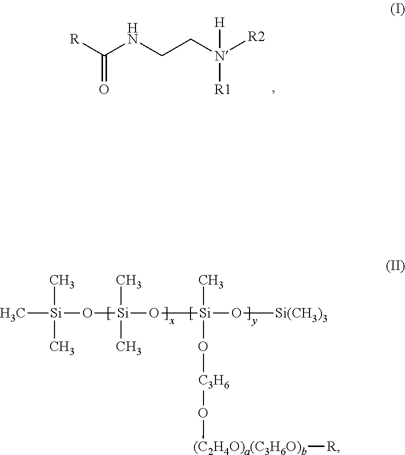

### FIELD OF THE INVENTION

The present invention generally relates to cosmetics, and more particularly relates to a cleaning agent including in a cosmetically acceptable carrier at least one anionic surfactant, at least one amphoteric and/or zwitterionic surfactant, at least one silicone and at least one cationic polymer.

### BACKGROUND OF THE INVENTION

Cosmetic cleaning agents, such as for example hair shampoos, are based on conventional anionic, amphoteric, zwitterionic, non-ionic and/or cationic surfactants.

Owing to their outstanding cleaning and foaming ability, anionic surfactants, optionally mixed with small amounts of co-surfactants, are predominantly used.

Such a commercial shampoo cleans the hair and removes sebaceous residues and/or residues of styling agents and other impurities from the surface of the hair and from the scalp.

However, the cleaning process also removes lipids and proteins from the hair and scalp, which can damage the hair structure and cause the scalp to dry out, particularly with frequent cleaning.

To eliminate these disadvantages, surfactants which are gentle and well tolerated have preferably been used in hair cleaning agents in recent years.

Although surfactants that are gentle per se are known, they do not yet satisfy all consumer requirements to an adequate degree.

Thus gentle surfactants are often not highly foaming and reduce the amount and quality of foam (creaminess and fine-pored nature of the foam). The incorporation of care substances into hair cleaning agents can further reinforce this effect.

Finally, sensory hair quality plays a prominent role among consumers with regard to acceptance of a shampoo.

Along with cleaning performance, the feel of the hair after washing, in both the wet and dry state, determines the satisfaction of a consumer with a cosmetic hair cleaning agent.

The critical factor in this perception is the sense of care, which can be described in particular by the feel, the combability, the shine and the softness of the hair. Furthermore, the hair should not appear heavy, nor should it be electrostatically charged.

There is therefore still a demand for active agents or for combinations of active agents for cosmetic cleaning preparations having good caring properties, an advantageous rheology profile and good foam properties.

It is therefore desirable to provide balanced, caring cleaning agents having good foam quality.

The cleaning agents should be suitable in particular for the gentle cleaning and care of the hair, and after application they should impart an improved combability and increased shine to the hair.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

It was found that cleaning agents based on anionic and special amphoteric/zwitterionic surfactants and on cationic polymers and silicones are outstandingly suitable. The corresponding cleaning agents care for and clean the hair and in combination with water form a creamy, fine-pored foam.

A cosmetic cleaning agent according to the present invention includes, in a cosmetically acceptable carrier, a) at least one anionic surfactant; b) at least one amphoteric and/or zwitterionic surfactant of the following formula (I)

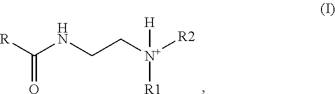

in which R denotes a straight-chain or branched, saturated or mono- or polyunsaturated alkyl or alkenyl residue having 8 to 24 carbon atoms, and R1 and R2 independently of one another denote the groups —(CH₂)ₙ—COOX, —(CH₂)ₙ—O—(CH₂)ₙ—COOX or —(CH₂)ₙ—OH, wherein n is an integer from 1 to 4 and X is an alkali, an alkaline-earth, an ammonium or an alkanolamine ion; c) at least one silicone of the following formula (II)

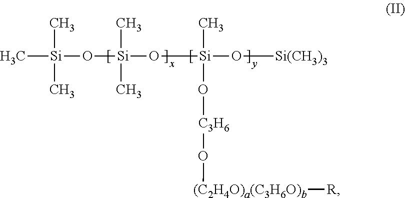

in which the residue R denotes a hydrogen atom, an alkyl group having 1 to 12 C atoms, an alkoxy group having 1 to 12 C atoms or a hydroxyl group, x denotes a number from 1 to 100, preferably from 20 to 30, y denotes a number from 1 to 20, preferably from 2 to 10, and a and b independently of one another denote numbers from 0 to 50, preferably from 10 to 30; and d) at least one cationic polymer selected from quaternized cellulose derivatives.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The invention therefore firstly provides a cosmetic cleaning agent including in a cosmetically acceptable carrier
a) at least one anionic surfactant,
b) at least one amphoteric and/or zwitterionic surfactant of the following formula (I)

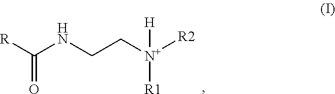

in which
R denotes a straight-chain or branched, saturated or mono- or polyunsaturated alkyl or alkenyl residue having 8 to 24 carbon atoms, and
R1 and R2 independently of one another denote the groups —(CH₂)ₙ—COOX, —(CH₂)ₙ—O—(CH₂)ₙ—COOX or —(CH₂)ₙ—OH, wherein n is an integer from 1 to 4 and X is an alkali, an alkaline-earth, an ammonium or an alkanolamine ion,
c) at least one silicone of the following formula (II)

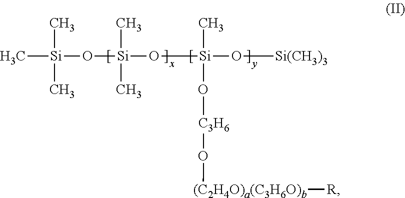

in which
the residue R denotes a hydrogen atom, an alkyl group having 1 to 12 C atoms, an alkoxy group having 1 to 12 C atoms or a hydroxyl group,
x denotes a number from 1 to 100, preferably from 20 to 30,
y denotes a number from 1 to 20, preferably from 2 to 10, and
a and b independently of one another denote numbers from 0 to 50, preferably from 10 to 30, and
d) at least one cationic polymer selected from quaternized cellulose derivatives.

The compositions according to the invention include components a) to d) in a cosmetically acceptable carrier. This is preferably aqueous or aqueous-alcoholic. The cosmetic carrier preferably includes at least 50 wt. %, more preferably at least 60 wt. % and particularly preferably at least 70 wt. % water.

The cosmetic carrier can moreover include 0.01 to 50 wt. %, preferably 0.05 to 40 wt. % and in particular 0.1 to 30 wt. % of at least one alcohol, which can be selected from ethanol, ethyl diglycol, 1-propanol, 2-propanol, isopropanol, 1,2-propylene glycol, glycerol, diglycerol, triglycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol, 1,2-hexanediol, 1,6-hexanediol, polyethylene glycols, sorbitol, sorbitan, benzyl alcohol, phenoxyethanol or mixtures of said alcohols.

The water-soluble alcohols are preferred.

Ethanol, ethyl diglycol, 1-propanol, 2-propanol, isopropanol, 1,2-propylene glycol, glycerol, benzyl alcohol and/or phenoxyethanol and mixtures of said alcohols are preferred in particular.

Suitable anionic surfactants a) are understood to include, for example:
linear alkyl sulfonates having 8 to 24 C atoms,
linear alpha-olefin sulfonates having 8 to 24 C atoms,
alkyl sulfates and alkyl polyglycol ether sulfates of formula R—(OCH₂—CH₂)ₓ—O—SO₃X, in which R preferably denotes a linear or branched, saturated or unsaturated alkyl group having 8 to 30 C atoms, x denotes 0 or a number from 1 to 12 and X denotes an alkali, an alkaline-earth, an ammonium or an alkanolamine ion,
sulfonates of unsaturated fatty acids having 8 to 24 C atoms and 1 to 6 double bonds.

Preferred anionic surfactants are alkyl sulfates and alkyl polyglycol ether sulfates of the aforementioned formula—preferably those including an alkyl group having 8 to 18 and in particular 10 to 16 C atoms along with 1 to 6 and in particular 2 to 4 ethylene oxide units. Further preferred anionic surfactants are straight-chain or branched alkyl sulfonates including an alkyl residue having 8 to 18 and in particular 10 to 16 C atoms.

The sodium, magnesium and/or triethanolamine salts of linear or branched lauryl, tridecyl and/or myristyl sulfates having a degree of ethoxylation from 2 to 4 are preferred in particular.

The anionic surfactant(s) can be used in the cleaning agents according to the invention—relative to their total weight—in an amount preferably from 6 to 18 wt. %, more preferably from 7 to 17 wt. %, particularly preferably from 8 to 15 wt. % and in particular from 9 to 13 wt. %.

Preferred amphoteric and/or zwitterionic surfactants of the aforementioned formula (I) are those in which the residue R predominantly denotes a straight-chain or branched, saturated or mono- or polyunsaturated alkyl residue having 8 to 20, more preferably 8 to 16 and in particular 8 to 12 C atoms.

Amphoteric and/or zwitterionic surfactants of the aforementioned formula (I) in which the residue R is derived from coconut oil are more preferred.

The surfactants known under the INCI names Sodium Cocoamphoacetate, Disodium Cocoamphodiacetate, Sodium Cocoamphopropionate and Disodium Cocoamphodipropionate, which are commercially available from a number of suppliers, are preferred in particular.

The amphoteric and/or zwitterionic surfactant(s) are used in the cleaning agents according to the invention—relative to their total weight—in an amount preferably from 0.5 to 5 wt. %, more preferably from 0.75 to 4.5 wt. %, particularly preferably from 1 to 4 wt. % and in particular from 1.5 to 3.5 wt. %.

Preferred silicones of the aforementioned formula (II) are those in which
the residue R denotes a hydrogen atom,
x denotes a number from 1 to 100, preferably from 20 to 30,
y denotes a number from 1 to 20, preferably from 2 to 10,
b denotes the number 0 and
a denotes a number from 10 to 30, preferably from 10 to 20 and in particular from 10 to 15.

Silicones of the aforementioned formula (II) which are particularly suitable for the cosmetic cleaning agents according to the invention are for example those which are known under the CTFA name Dimethicone Copolyol and/or under the INCI name PEG-a/PPG-b Dimethicone, where a and b have the same meaning as in formula (II).

Suitable silicones are commercially available for example under the names Silsoft® (Momentive), Silwet® (General Electric), Dow Corning® (Dow), Belsil® (Wacker) and Abil® (Goldschmidt).

Preferred commercial products which include silicones of the aforementioned formula (II) with the CTFA name Dimethicone Copolyol and/or the INCI name PEG-a/PPG-b Dimethicone and which can be used in the cleaning agents according to the invention are for example Silsoft® 895 (Momentive), Silwet® L 722 (General Electric), Silwet® 7001 (General Electric), Silwet® 7605 (General Electric), Silwet® 7500 (General Electric), Dow Corning® 190 (Dow), Dow Corning® 193 (Dow), Dow Corning® 3225 (Dow), Belsil® DMC 6031 (Wacker) and/or Abil® 8842 (Goldschmidt).

The silicone(s) of the aforementioned formula (II) are used in the cleaning agents according to the invention—relative to their total weight—preferably in an amount from 0.05 to 3 wt. %, more preferably from 0.1 to 2.75 wt. %, particularly preferably from 0.2 to 2.5 wt. % and in particular from 0.25 to 2 wt. %.

Suitable cationic polymers d) are understood to be quatemized cellulose derivatives.

Preferred quaternary cellulose derivatives are polymeric quaternary ammonium salts formed in the reaction of hydroxyethyl cellulose with trimethylammonium-substituted epoxides, for example the cationic polymers known under the INCI name Polyquaternium-10.

Polyquaternium-10 is commercially available from a number of suppliers.

The polymers known under the trade names Celquat®, Polymer JR® or Polymer LR® for example are suitable for the cleaning agents according to the invention.

Polymer JR® 400 from Amerchol is suitable in particular.

The cationic polymer(s) are used in the cleaning agents according to the invention—relative to their total weight—preferably in an amount from 0.01 to 3 wt. %, more preferably from 0.02 to 2 wt. %, particularly preferably from 0.03 to 1.5 wt. % and in particular from 0.05 to 1 wt. %.

In a first particularly preferred embodiment cleaning agents according to the invention include—relative to their total weight—
a) 6 to 18 wt. %, preferably from 7 to 17 wt. %, of at least one alkyl (ether) sulfate or sulfonate,
b) 0.5 to 5 wt. %, preferably from 0.75 to 4.5 wt. %, of at least one zwitterionic and/or amphoteric surfactant of the aforementioned formula (I), in which R predominantly denotes a straight-chain or branched, saturated or mono- or polyunsaturated alkyl residue having 8 to 20, more preferably from 8 to 16 and in particular 8 to 12 C atoms,
c) 0.05 to 3 wt. %, preferably from 0.1 to 2.75 wt. %, of at least one silicone of formula (II), in which
   the residue R denotes a hydrogen atom, an alkyl group having 1 to 12 C atoms, an alkoxy group having 1 to 12 C atoms or a hydroxyl group,
   x denotes a number from 1 to 100, preferably from 20 to 30,
   y denotes a number from 1 to 20, preferably from 2 to 10, and
   a and b independently of one another denote numbers from 0 to 50, preferably from 10 to 30, and
d) 0.01 to 3 wt. %, preferably from 0.02 to 2 wt. %, of at least one cationic polymer which is a quaternized cellulose derivative.

Within this embodiment it is particularly preferable if cleaning agents according to the invention include—relative to their total weight
a) 8 to 15 wt. % of at least one alkyl sulfate and/or alkyl polyglycol ether sulfate having an alkyl group with 8 to 18 and in particular 10 to 16 C atoms and with 1 to 6 and in particular 2 to 4 ethylene oxide units,
b) 1 to 4 wt. % of at least one of the surfactants known under the INCI names Sodium Cocoamphoacetate, Disodium Cocoamphodiacetate, Sodium Cocoamphopropionate and Disodium Cocoamphodipropionate,
c) 0.2 to 2.5 wt. % of at least one silicone of formula (II), in which
   the residue R denotes a hydrogen atom,
   x denotes a number from 1 to 100, preferably from 20 to 30,
   y denotes a number from 1 to 20, preferably from 2 to 10,
   b denotes the number 0 and
   a denotes a number from 10 to 30, preferably from 10 to 20 and in particular from 10 to 15, and
d) 0.03 to 1.5 wt. % of at least one cationic polymer known under the INCI name Polyquaternium-10.

Cosmetic cleaning agents that are preferred in particular within this embodiment include—relative to their total weight
a) 9 to 13 wt. % of sodium, magnesium and/or triethanolamine salts of linear or branched lauryl, tridecyl and/or myristyl sulfates having a degree of ethoxylation from 2 to 4,
b) 1.5 to 3.5 wt. % of at least one of the surfactants known under the INCI names Sodium Cocoamphoacetate, Disodium Cocoamphodiacetate, Sodium Cocoamphopropionate and Disodium Cocoamphodipropionate,
c) 0.25 to 2 wt. % of at least one silicone of the aforementioned formula (II) known under the CTFA name Dimethicone Copolyol and/or under the INCI name PEG-a/PPG-b Dimethicone, and
d) 0.05 to 1 wt. % of at least one cationic polymer known under the INCI name Polyquaternium-10.

It has been found that the caring properties of the cleaning agents according to the invention can be further increased if one or more vitamins, vitamin derivatives and/or vitamin precursors are added to them.

Therefore, in a further preferred embodiment, cleaning agents according to the invention include—relative to their total weight—preferably 0.001 to 5 wt. %, more preferably 0.002 to 4 wt. % and particularly preferably 0.0025 to 3 wt. % of at least one vitamin, provitamin and/or vitamin precursor (e)).

Suitable vitamins, provitamins and vitamin precursors e) are preferably vitamins, provitamins and vitamin precursors that are assigned to groups A, B, C, E, F and H. Vitamin A: The group of substances classed as vitamin A includes retinol (vitamin A₁) and 3,4-didehydroretinol (vitamin A₂). β-Carotene is the retinol provitamin. Suitable vitamin A components are for example vitamin A acid and esters thereof, vitamin A aldehyde and vitamin A alcohol and esters thereof such as the palmitate and acetate.
Vitamin B: The vitamin B group or vitamin B complex includes inter alia
   Vitamin B₁ (thiamine)
   Vitamin B₂ (riboflavin)
   Vitamin B₃. The compounds nicotinic acid and nicotinic acid amide (niacinamide) are often included under this term.
   Vitamin B₅ (pantothenic acid, panthenol and pantolactone). Within the context of this group panthenol and/or pantolactone is preferably used in the agents according to the invention. Derivatives of panthenol which can be used are in particular the esters and ethers of panthenol as well as cationically derivatized panthenols. Individual representatives are for example panthenol triacetate, panthenol monoethyl ether and the monoacetate thereof, and cationic panthenol derivatives.
   Vitamin B₆ (pyridoxine as well as pyridoxamine and pyridoxal).
Vitamin C (ascorbic acid): Use in the form of the palmitic acid ester, glucosides or phosphates can be preferred. Use in combination with tocopherols can likewise be preferred.
Vitamin E (tocopherols, in particular α-tocopherol): These include tocopherol and derivatives thereof, which are understood in particular to be the esters such as the acetate, the nicotinate, the phosphate and the succinate.
Vitamin F: The term “vitamin F” is conventionally understood to mean essential fatty acids, in particular linoleic acid, linolenic acid and arachidonic acid.
Vitamin H: Vitamin H is the name given to the compound (3aS,4S,6aR)-2-oxohexahydrothienol[3,4-d]-imidazole-4-valeric acid, although this is now more widely known by the trivial name biotin.

Preferred agents according to the invention include at least one vitamin, provitamin or vitamin precursor from the aforementioned groups A, B, E and H.

Particularly preferred agents according to the invention include at least one vitamin, provitamin or vitamin precursor from the B group.

Agents according to the invention that are preferred in particular include niacinamide, panthenol, pantolactone and/or pyridoxine.

In a second particularly preferred embodiment cleaning agents according to the invention include—relative to their total weight
a) 6 to 18 wt. %, preferably from 7 to 17 wt. %, of at least one alkyl (ether) sulfate or sulfonate,
b) 0.5 to 5 wt. %, preferably from 0.75 to 4.5 wt. %, of at least one zwitterionic and/or amphoteric surfactant of the aforementioned formula (I), in which R predominantly denotes a straight-chain or branched, saturated or mono- or polyunsaturated alkyl residue having 8 to 20, more preferably from 8 to 16 and in particular 8 to 12 C atoms,
c) 0.05 to 3 wt. %, preferably from 0.1 to 2.75 wt. %, of at least one silicone of formula (II), in which
   the residue R denotes a hydrogen atom, an alkyl group having 1 to 12 C atoms, an alkoxy group having 1 to 12 C atoms or a hydroxyl group,
   x denotes a number from 1 to 100, preferably from 20 to 30,
   y denotes a number from 1 to 20, preferably from 2 to 10, and
   a and b independently of one another denote numbers from 0 to 50, preferably from 10 to 30,
d) 0.01 to 3 wt. %, preferably from 0.02 to 2 wt. %, of at least one cationic polymer which is a quaternized cellulose derivative, and
e) 0.001 to 5 wt. % of at least one vitamin, vitamin precursor or vitamin derivative.

Within this embodiment it is particularly preferable if cleaning agents according to the invention include—relative to their total weight
a) 8 to 15 wt. % of at least one alkyl sulfate and/or alkyl polyglycol ether sulfate having an alkyl group with 8 to 18 and in particular 10 to 16 C atoms and with 1 to 6 and in particular 2 to 4 ethylene oxide units,
b) 1 to 4 wt. % of at least one of the surfactants known under the INCI names Sodium Cocoamphoacetate, Disodium Cocoamphodiacetate, Sodium Cocoamphopropionate and Disodium Cocoamphodipropionate,
c) 0.2 to 2.5 wt. % of at least one silicone of formula (II), in which
   the residue R denotes a hydrogen atom,
   x denotes a number from 1 to 100, preferably from 20 to 30,
   y denotes a number from 1 to 20, preferably from 2 to 10,
   b denotes the number 0 and
   a denotes a number from 10 to 30, preferably from 10 to 20 and in particular from 10 to 15,
d) 0.03 to 1.5 wt. % of at least one cationic polymer known under the INCI name Polyquaternium-10, and
e) 0.002 to 4 wt. % of at least one vitamin, provitamin or vitamin precursor from the aforementioned groups A, B, E and H.

Cosmetic cleaning agents that are preferred in particular within this embodiment include—relative to their total weight
a) 9 to 13 wt. % of sodium, magnesium and/or triethanolamine salts of linear or branched lauryl, tridecyl and/or myristyl sulfates having a degree of ethoxylation from 2 to 4,
b) 1.5 to 3.5 wt. % of at least one of the surfactants known under the INCI names Sodium Cocoamphoacetate, Disodium Cocoamphodiacetate, Sodium Cocoamphopropionate and Disodium Cocoamphodipropionate,
c) 0.25 to 2 wt. % of at least one silicone of the aforementioned formula (II) known under the CTFA name Dimethicone Copolyol and/or under the INCI name PEG-a/PPG-b Dimethicone,
d) 0.05 to 1 wt. % of at least one cationic polymer known under the INCI name Polyquaternium-10, and
e) 0.0025 to 3 wt. % of at least one vitamin, provitamin or vitamin precursor of the B group.

In addition to the required and preferred components that have already been mentioned, the cleaning agents according to the invention can include further additives that impart advantageous properties to them, for example further—in particular non-ionic—surfactants and/or emulsifiers (which differ from the surfactants a) and b)), further cationic polymers (which differ from d)), oils, fats and/or waxes, protein hydrolysates, pearlescent agents and/or plant extracts.

Suitable non-ionic surfactants can be used in the agents according to the invention preferably in amounts from 0 to 20 wt. %, more preferably from 0.25 to 17.5 wt. %, particularly preferably from 0.5 to 15 wt. % and in particular from 1 to 10 wt. %, the stated amounts relating to the total weight of the cleaning agent.

The suitable non-ionic surfactants/emulsifiers include for example
C₈-C₃₀ fatty acid monoesters and diesters of addition products of 1 to 30 mol of ethylene oxide with glycerol,
amine oxides,
addition products of 2 to 50 mol of ethylene oxide and/or 0 to 5 mol of propylene oxide with linear and branched fatty alcohols having 8 to 30 C atoms, with fatty acids having 8 to 30 C atoms and with alkyl phenols having 8 to 15 C atoms in the alkyl group,
sorbitan fatty acid esters and addition products of ethylene oxide with sorbitan fatty acid esters such as for example polysorbates,
sugar fatty acid esters and addition products of ethylene oxide with sugar fatty acid esters,
addition products of ethylene oxide with fatty acid alkanolamides and fatty amines, and/or
alkyl polyglucosides.

If a non-ionic surfactant is used as the further surfactant in the agents according to the invention, alkyl oligoglucosides, in particular alkyl oligoglucosides based on hydrogenated C_{12/14} coconut alcohol with a DP of 1 to 3, such as are available commercially for example under the INCI name Coco-Glucosides, are preferred.

Further preferred non-ionic surfactants are the C₈-C₃₀ fatty acid mono- and diesters of addition products of 1 to 30 mol of ethylene oxide with glycerol. The C₁₀-C₁₆ fatty acid mono- and diesters of addition products of 1 to 10 mol of ethylene oxide with glycerol are particularly preferred. The product known under the INCI name PEG-7 Glyceryl Cocoate is preferred in particular.

Suitable further cationic polymers which differ from the polymers d) can be used in the cleaning agents according to the invention—relative to their total weight—preferably in amounts from 0.01 to 2 wt. %, preferably from 0.02 to 1 wt. % and in particular in amounts from 0.025 to 0.8 wt. %.

Suitable further cationic polymers are preferably polymers having groups in the main and/or side chain which can be “temporarily” or “permanently” cationic. Polymers which have a cationic group irrespective of the pH of the agent are described as “permanently cationic”. These are generally polymers including a quaternary nitrogen atom, in the form of an ammonium group for example. Preferred cationic groups include quaternary ammonium groups.

Preferred further cationic polymers are for example
hydrophobically modified cellulose derivatives, for example the cationic polymers sold under the trade name SoftCat®,
cationic alkyl polyglycosides,
cationized honey, for example the commercial product Honeyquat® 50,
cationic guar derivatives, such as in particular the products sold under the trade names Cosmedia® Guar and Jaguar®,
polymeric dimethyldiallyl ammonium salts and copolymers thereof with esters and amides of acrylic acid and methacrylic acid. The products available commercially under the names Merquat® 100 (poly(dimethyl diallyl ammonium chloride)) and Merquat® 550 (dimethyl diallyl ammonium chloride-acrylamide copolymer) are examples of such cationic polymers,
copolymers of vinylpyrrolidone with quaternized derivatives of dialkyl aminoalkyl acrylate and methacrylate, such as for example diethyl sulfate-quaternized vinylpyrrolidone-dimethyl aminoethyl methacrylate copolymers. Such compounds are available commercially under the names Gafquat® 734 and Gafquat® 755,
vinylpyrrolidone-vinylimidazolium methochloride copolymers, such as are sold under the names Luviquat® FC 370, FC 550, FC 905 and HM 552,
quaternized polyvinyl alcohol, as well as the polymers known under the names
Polyquaternium-2, Polyquaternium-17, Polyquaternium-18, Polyquaternium-27, Polyquaternium-32, Polyquaternium-37, Polyquaternium-67, Polyquaternium-74 and Polyquaternium-89.

The polymers known under the name Polyquaternium-24 (commercial product e.g. Quatrisoft® LM 200) can likewise be used as cationic polymers. Likewise suitable for use according to the invention are the copolymers of vinylpyrrolidone, such as are available as the commercial products Copolymer 845 (manufacturer: ISP), Gaffix® VC 713 (manufacturer: ISP), Gafquat® ASCP 1011, Gafquat® HS 110, Luviquat® 8155 and Luviquat® MS 370.

Further cationic polymers according to the invention are the “temporarily cationic” polymers. These polymers conventionally include an amino group that at certain pH values takes the form of a quaternary ammonium group and is therefore cationic. Chitosan and derivatives thereof, such as are widely available commercially under the trade names Hydagen® CMF, Hydagen® HCMF, Kytamer® PC and Chitolam® NB/101, for example, are preferred. Chitosans are deacetylated chitins, which are commercially available in various degrees of deacetylation and in various degrees of decomposition (molecular weights).

Particularly preferred further cationic polymers are cationic guar derivatives and/or cationic polymers based on acrylic acid (derivatives), which can be selected in particular from the polymers known under the INCI names Jaguar® or N-Hance®, Polyquaternium-6, Polyquaternium-7, Polyquaternium-67, Polyquaternium-74 and/or Polyquaternium-89.

Suitable oil and/or fat components are preferably selected from mineral, natural and synthetic oil components and/or fats.

They can be used in the cleaning agents according to the invention preferably in an amount from 0.001 to 10 wt. %, more preferably from 0.005 to 7.5 wt. % and in particular from 0.01 to 5 wt. %, the stated amounts relating to the total weight of the final cleaning agent.

Triglycerides and mixtures of triglycerides are conventionally used as natural (vegetable) oils. Preferred natural oils are coconut oil, (sweet) almond oil, walnut oil, peach kernel oil, apricot kernel oil, avocado oil, tea tree oil, soybean oil, sesame oil, sunflower oil, tsubaki oil, evening primrose oil, rice bran oil, palm kernel oil, mango kernel oil, lady's smock oil, thistle oil, macadamia nut oil, grape seed oil, amaranth seed oil, argan oil, bamboo oil, olive oil, wheatgerm oil, pumpkin seed oil, mallow oil, hazelnut oil, safflower oil, canola oil, sasanqua oil, jojoba oil, rambutan oil, cocoa butter and shea butter.

Suitable mineral oils are in particular mineral oils, paraffin and isoparaffin oils and synthetic hydrocarbons. One example of a suitable hydrocarbon is the commercially available 1,3-di-(2-ethylhexyl)cyclohexane (Cetiol® S), for example.

A dialkyl ether can moreover serve as the oil component.

Suitable dialkyl ethers are in particular di-n-alkyl ethers having in total between 12 and 36 C atoms, in particular between 12 and 24 C atoms, such as for example di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether and also di-tert-butyl ether, diisopentyl ether, di-3-ethyl decyl ether, tert-butyl-n-octyl ether, isopentyl-n-octyl ether and 2-methyl pentyl-n-octyl ether.

Di-n-octyl ether, which is commercially available under the name Cetiol® OE, is particularly preferred.

Silicone compounds which differ from the silicone compounds c) are suitable as synthetic oils.

Suitable silicones (which differ from the silicone c)) can be selected from:
(i) polyalkylsiloxanes, polyarylsiloxanes, polyalkylarylsiloxanes, which are volatile or non-volatile, straight-chain, branched or cyclic, crosslinked or uncrosslinked;
(ii) polysiloxanes which in their general structure include one or more organofunctional groups, which are selected from:
   a) substituted or unsubstituted aminated groups;
   b) (per)fluorinated groups;
   c) thiol groups;
   d) carboxylate groups;
   e) hydroxylated groups;
   f) acyloxyalkyl groups;
   g) amphoteric groups;
   h) bisulfite groups;
   i) hydroxyacyl amino groups;
   j) carboxyl groups
   k) sulfonic acid groups; and
   l) sulfate or thiosulfate groups;
(iii) grafted silicone polymers having a non-silicone-containing, organic framework consisting of an organic main chain formed from organic monomers including no silicone, onto which at least one polysiloxane macromer has been grafted in the chain and optionally on at least one chain end;
(iv) grafted silicone polymers having a polysiloxane framework, onto which non-silicone-containing, organic monomers have been grafted, which have a polysiloxane main chain onto which at least one organic macromer including no silicone has been grafted in the chain and optionally on at least one of its ends;
(v) or mixtures thereof.

Fats are understood to be fatty acids, fatty alcohols and natural and synthetic waxes, which can be present both in solid form and in liquid form in aqueous dispersion.

Linear and/or branched, saturated and/or unsaturated fatty acids having 6 to 30 carbon atoms can be used as fatty acids. Fatty acids having 10 to 22 carbon atoms are preferred. Examples which can be cited include the isostearic acids, such as the commercial products Emersol® 871 and Emersol® 875, and isopalmitic acids such as the commercial product Edenor® IP 95, as well as all further fatty acids sold under the Edenor® trade names (Cognis). Further typical examples of such fatty acids are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, eicosanoic acid, gadoleic acid, docosanoic acid and erucic acid and technical mixtures thereof.

The fatty acid cuts obtainable from coconut oil or palm oil are conventionally particularly preferred; as a rule the use of stearic acid is preferred in particular.

Saturated, mono- or polyunsaturated, branched or unbranched fatty alcohols having C₆ to C₃₀, preferably C₁₀ to C₂₂ and most particularly preferably C₁₂ to C₂₂ carbon atoms can be used as fatty alcohols. For example, decanol, octanol, octenol, dodecenol, decenol, octadienol, dodecadienol, decadienol, oleyl alcohol, erucic alcohol, ricinol alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol, as well as the Guerbet alcohols thereof, can be used, wherein this list is intended to be of an exemplary and non-limiting nature. However, the fatty alcohols derive from preferably natural fatty acids, wherein it can conventionally be assumed that they are obtained from the esters of fatty acids by reduction. Fatty alcohol cuts which are produced by reduction of naturally occurring triglycerides such as beef fat, palm oil, groundnut oil, colza oil, cottonseed oil, soybean oil, sunflower oil and linseed oil or from fatty acid esters formed from the transesterification products thereof with corresponding alcohols and which thus represent a mixture of different fatty alcohols, can likewise be used. Such substances are available commercially for example under the names Stenol®, e.g. Stenol® 1618, or Lanette®, e.g. Lanette® O, or Lorol®, e.g. Lorol® C8, Lorol® C14, Lorol® C18, Lorol® C8-18, HD-Ocenol®, Crodacol®, e.g. Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. Wool wax alcohols can of course also be used according to the invention, such as are available commercially for example under the names Corona®, White Swan®, Coronet® or Fluilan®.

Solid paraffins or isoparaffins, carnauba waxes, beeswaxes, candelilla waxes, ozocerites, ceresin, spermaceti wax, sunflower wax, fruit waxes such as for example apple wax or citrus wax, PE or PP microwaxes can be used as natural or synthetic waxes. Such waxes are available for example via Kahl & Co., Trittau.

Further fats are, for example
ester oils. Ester oils are understood to be the esters of C₆-C₃₀ fatty acids with C₂-C₃₀ fatty alcohols. The monoesters of fatty acids with alcohols having 2 to 24 C atoms are preferred. Examples of fatty acid components used in the esters are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, eicosanoic acid, gadoleic acid, docosanoic acid and erucic acid and technical mixtures thereof. Examples of the fatty alcohol components in the ester oils are isopropyl alcohol, hexanol, octanol, 2-ethylhexyl alcohol, decanol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and technical mixtures thereof. Isopropyl myristate (Rilanit® IPM), isononanoic acid C₁₆-₁₈ alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V) are particularly preferred.
dicarboxylic acid esters such as di-n-butyl adipate, di-(2-ethylhexyl)adipate, di-(2-ethylhexyl)succinate and diisotridecyl acelate and also diol esters such as ethylene glycol dioleate, ethylene glycol diisotridecanoate, propylene glycol di-(2-ethyl hexanoate), propylene glycol diisostearate, propylene glycol dipelargonate, butanediol diisostearate, neopentyl glycol dicaprylate,
symmetrical, asymmetrical or cyclic esters of carbonic acid with fatty alcohols,
glycerol carbonate or dicaprylyl carbonate (Cetiol® CC),
ethoxylated or non-ethoxylated mono-, di- and tri-fatty acid esters of saturated and/or unsaturated linear and/or branched fatty acids with glycerol, such as for example Monomuls® 90-O18, Monomuls® 90-L12, Cetiol® HE or Cutina® MD.

Suitable protein hydrolysates which can be used in the cleaning agents according to the invention are preferably of plant, animal or marine origin. They can be used in the agents according to the invention preferably in an amount from 0.01 to 10 wt. %, more preferably from 0.25 to 7.5 wt. % and in particular from 0.05 to 5 wt. %, the stated amounts relating to the total weight of the final cleaning agent.

Suitable animal protein hydrolysates are for example elastin, collagen, keratin, silk and/or milk protein hydrolysates, which can also be present in the form of salts.

Such products are sold for example under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

Suitable protein hydrolysates of plant origin are for example soy, almond, rice, pea, potato, rapeseed and/or wheat protein hydrolysates.

Such products are available for example under the trademarks Gluadin® (Cognis), DiaMin® (Diamalt), Lexein® (Inolex) and Crotein® (Croda).

The suitable protein hydrolysates of marine origin include for example collagen hydrolysates of fish or algae and protein hydrolysates of mussels or pearl hydrolysates. Examples of suitable pearl hydrolysates are the commercial products Pearl Protein Extract BG® or Crodarom® Pearl.

Cationized protein hydrolysates can also be used, wherein the underlying protein hydrolysate can originate from the animal, plant and/or marine sources described above.

Cationic protein hydrolysates are moreover understood to include quaternized amino acids and mixtures thereof. The quaternization of protein hydrolysates or amino acids is frequently performed using quaternary ammonium salts such as for example N,N-dimethyl-N-(n-alkyl)-N-(2-hydroxy-3-chloro-n-propyl)ammonium halides.

The cationic protein hydrolysates can moreover also be further derivatized.

Typical examples of suitable cationic protein hydrolysates and/or derivatives are the commercially available products known under the following INCI names: Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCI, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxyproypltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quatemium-79 Hydrolyzed Keratin, Quaternium-79 Hydrolyzed Milk Protein, Quaternium-79 Hydrolyzed Silk, Quaternium-79 Hydrolyzed Soy Protein, Quaternium-79 Hydrolyzed Wheat Protein.

Suitable pearlescent agents according to the invention are for example
glycol distearic acid esters,
C₈-C₃₀ fatty acid monoglycol esters and/or
mica pigments coated with titanium dioxide,
   such as are available for example under the trade names Rewopal®, Genapol® PMS, Cutina® EGMS, Timiron®, Colorona® and Euperlan®.

The pearlescent agent(s) can be used in the cleaning agents according to the invention—relative to their weight—preferably in amounts from 0.01 to 3 wt. %, more preferably from 0.025 to 2 wt. % and particularly preferably from 0.05 to 1 wt. %.

Suitable plant extracts are understood to be extracts which can be produced from all parts of a plant.

These extracts are conventionally produced by extraction of the entire plant. It can also be preferable in individual cases, however, to produce the extracts exclusively from flowers and/or leaves of the plant.

The extracts from green tea, oak bark, stinging nettle, witch hazel, hops, chamomile, burdock, horsetail, whitethorn, lime blossom, lychee, almond, aloe vera, pine, horse chestnut, sandalwood, juniper, coconut, mango, apricot, lemon, wheat, kiwi, melon, orange, grapefruit, sage, rosemary, birch, mallow, lady's smock, wild thyme, yarrow, thyme, melissa, restharrow, coltsfoot, marshmallow, ginseng, ginger root, Echinacea purpurea, Olea europaea, Foeniculum vulgaris and Apium graveolens are preferred above all according to the invention.

Water, alcohols and mixtures thereof can be used as extracting agents to produce the cited plant extracts. Of the alcohols, low alcohols such as ethanol and isopropanol, but in particular polyhydric alcohols such as ethylene glycol and propylene glycol, are preferred, both as the sole extracting agent and mixed with water. Plant extracts based on water/propylene glycol in the ratio 1:10 to 10:1 have proved to be particularly suitable.

The plant extracts can be used according to the invention in both pure and diluted form. If they are used in diluted form they conventionally include approximately 2 to 80 wt. % of active substance and as the solvent the extracting agent or mixture of extracting agents used to obtain them.

Preferred embodiments of the cosmetic cleaning agents according to the invention are shampoos, shower washes, shower gels, hair rinses, hair masks, aftershaves and/or deodorants. Cleaning agents according to the invention which serve to clean the hair and scalp are preferred in particular.

In order to achieve maximum gentleness combined with effective cleaning and care, it is preferable for the cleaning agents according to the invention to have a pH in the range from 4 to 5.7, more preferably from 4.2 to 5.5 and in particular from 4.5 to 5.3.

In addition to the required components according to the invention and the further aforementioned preferred components, the cleaning preparations according to the invention can include further components known to the person skilled in the art for such cosmetic agents.

These include for example:
texturizing agents such as maleic acid and lactic acid,
active agents to improve the fiber structure, in particular mono-, di- and oligosaccharides such as for example glucose, galactose, fructose, fruit sugar and lactose,
dyes to color the agent,
anti-dandruff active agents such as piroctone olamine, zinc omadine, zinc pyrithione and climbazole,
further substances to adjust the pH, such as for example α- and β-hydroxycarboxylic acids,
active agents such as allantoin and bisabolol,
complexing agents such as EDTA, NTA, β-alanine diacetic acid and phosphonic acids,
propellants such as propane-butane mixtures, N₂O, dimethyl ether, CO₂ and air,
consistency modifiers such as sugar esters, polyol esters or polyol alkyl ethers,
preservatives, such as for example sodium benzoate or salicylic acid,
viscosity adjusters such as salts (NaCl).

The cleaning agents according to the invention have outstanding properties.

In addition to outstanding cleaning of skin and hair, they form a soft foam which can easily be distributed, and after rinsing they leave behind a pleasant feel on the application surface.

The agents according to the invention are suitable in particular for cleaning and/or caring for the hair and/or scalp, since it was observed that both the hair and the skin are less stressed and dried out as a result of the use of the cleaning agents according to the invention.

It was also observed that with regular use the agents according to the invention strengthen the hair structure.

Hair treated with the agents according to the invention has in particular an improved combability and an improved shine.

The invention therefore secondly provides the use of an agent according to the invention for cleaning and caring for hair, in particular for improving the combability and the shine of hair and for strengthening the hair structure.

### EXAMPLES

The table below includes six shampoo test formulations, shampoos 1, 5 and 6 being shampoos according to the invention.

The stated amounts in the table relate to percentages by weight.

| | 1 | 2 | 3 | 4 | 5 | 6 |
|---|---|---|---|---|---|---|
| Sodium laureth sulfate | 11 | 11 | 11 | 11 | 11 | 11 |
| Cocamidopropyl betaine | | 2.5 | | | | |
| Disodium cocoamphodiacetate | 2.5 | | 2.5 | 2.5 | 2.5 | 2.5 |
| Polyquaternium-10 | 0.45 | 0.45 | 0.45 | | 0.45 | 0.45 |
| Guar hydroxypropyl trimonium chloride | | | | 0.45 | | |
| PEG-12 dimethicone | 0.8 | 0.8 | | 0.8 | 0.8 | 0.8 |
| Amodimethicone | | | 0.8 | | | |
| Panthenol | 0.3 | 0.3 | 0.3 | 0.3 | | 0.3 |
| Niacinamide | 0.25 | 0.25 | 0.25 | 0.25 | | 0.25 |
| Solubilizer | 0.4 | 0.4 | 0.4 | 0.4 | 0.4 | 0.4 |
| Perfume | 0.4 | 0.4 | 0.4 | 0.4 | 0.4 | 0.4 |
| Pearlescent agent | 2.5 | 2.5 | 2.5 | 2.5 | 2.5 | 2.5 |
| Acidulants, thickening and/or stabilizing | qs | qs | qs | qs | qs | qs |
| agents, preservatives | | | | | | |
| Water | to | to | to | to | to | to |
| | 100 | 100 | 100 | 100 | 100 | 100 |
| pH | 5.0 | 5.0 | 5.0 | 5.0 | 5.0 | 6.5 |
| Reduction in wet combability [%] | 56 | 41 | 32 | 58 | 54 | 57 |
| Increase in shine [%] | 92 | 76 | 88 | 73 | 93 | 89 |
| Structural improvement of damaged hair | 68 | 49 | 46 | 54 | 44 | 51 |
| (from DSC measurement) [%] | | | | | | |

The table shows that compositions 1, 5 and 6 according to the invention improve the combability and shine of the hair as compared with comparative compositions 2, 3 and 4. Compositions according to the invention also strengthen the structure of damaged hair, wherein the effect is greatest in the compositions which, in addition to the active agent combination according to the invention, include vitamins and have a low pH of about 5 (example 1).

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic cleaning agent, including in a cosmetically acceptable carrier
a) at least one anionic surfactant,
b) at least one amphoteric and/or zwitterionic surfactant of the following formula (I) 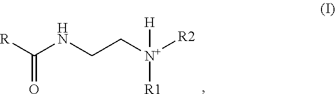
in which
R denotes a straight-chain or branched, saturated or mono- or polyunsaturated alkyl or alkenyl residue having 8 to 24 carbon atoms, and
R1 and R2 independently of one another denote the groups —(CH₂)ₙ—COOX, —(CH₂)ₙ—O—(CH₂)ₙ—COOX or —(CH₂)ₙ—OH, wherein n is an integer from 1 to 4 and X is an alkali, an alkaline-earth, an ammonium or an alkanolamine ion,
c) at least one silicone of the following formula (II) 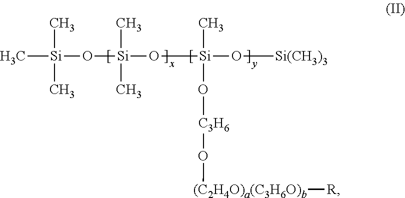
in which
the residue R denotes a hydrogen atom, an alkyl group having 1 to 12 C atoms, an alkoxy group having 1 to 12 C atoms or a hydroxyl group,
x denotes a number from 1 to 100,
y denotes a number from 1 to 20, and
a and b independently of one another denote numbers from 0 to 50, and
d) at least one cationic polymer selected from quaternized cellulose derivatives.

2. The cosmetic cleaning agent according to claim 1, comprising:
6 to 18 wt. % of at least one anionic surfactant a),
0.5 to 5 wt. % of at least one amphoteric and/or zwitterionic surfactant of formula (I),
0.05 to 3 wt. % of at least one silicone of formula (II), and
0.01 to 3 wt. % of at least one cationic polymer d),
wherein the stated amounts relate to the total weight of the cleaning agent.

3. The cosmetic cleaning agent according to claim 1, wherein the anionic surfactant(s) is (are) selected from the group of formula R—(OCH₂—CH₂)ₓ—OSO₃X, in which R denotes a linear or branched, saturated or unsaturated alkyl group having 8 to 30 C atoms, x denotes 0 or a number from 1 to 12 and X denotes an alkali, an alkaline-earth, an ammonium or an alkanolamine ion,

4. The cosmetic cleaning agent according to claim 1, wherein the amphoteric and/or zwitterionic surfactant(s) of formula (I) is (are) at least one surfactant selected from the group consisting of Sodium Cocoamphoacetate, Disodium Cocoamphodiacetate, Sodium Cocoamphopropionate and Disodium Cocoamphodipropionate.

5. The cosmetic cleaning agent according to claim 1, wherein in formula (II)
the residue R denotes a hydrogen atom,
x and y have the meaning given in claim 1,
b denotes the number 0, and
a denotes a number from 10 to 30, preferably from 10 to 20.

6. The cosmetic cleaning agent according to claim 1, wherein the cationic polymer(s) is (are) one or more polymers selected from the group consisting of the polymers known under the INCI name Polyquaternium-10.

7. The cosmetic cleaning agent according to claim 1, further comprising 0.001 to 5 wt. % of at least one vitamin, provitamin and/or vitamin derivative.

8. The cosmetic cleaning agent according to claim 7, the at least one vitamin, provitamin or vitamin derivative is selected from the group consisting of the B group.

9. The cosmetic cleaning agent according to claim 1, having a pH in the range from 4 to 5.7.
